(19) United States
(12) Patent Application Publication
(10) Pub. No.: US 2014/0371623 A1
(43) Pub. Date: Dec. 18, 2014

(54) ELECTROPHYSIOLOGY-BASED COCHLEA DEAD REGION DETECTION APPARATUS AND INFORMATION PROVISION METHOD FOR DETECTING COCHLEA DEAD REGION USING THE SAME

(71) Applicants: Sung Hwa Hong, Seocho-gu Seoul (KR); See Youn Kwon, Gangnam-gu Seoul (KR)

(72) Inventors: Sung Hwa Hong, Seocho-gu Seoul (KR); See Youn Kwon, Gangnam-gu Seoul (KR)

(73) Assignee: SAMSUNG LIFE PUBLIC WELFARE FOUNDATION, Yongsan-gu Seoul (KR)

(21) Appl. No.: 13/995,013

(22) PCT Filed: Mar. 15, 2011
(86) PCT No.: PCT/KR2011/001806

§ 371 Date: Aug. 9, 2013

(30) Foreign Application Priority Data
Dec. 17, 2010 (KR) 10-2010-0129709

(51) Int. Cl. A61B 5/12

(52) U.S. Cl. CPC A61B 5/125
USPC 600/559

(57) ABSTRACT

An apparatus for objectively detecting a cochlea dead region based on electrophysiology and an information provision method for detecting a cochlea dead region using the apparatus. A cochlea dead region detection apparatus including a control unit is also disclosed. The control unit includes a stimulus generation unit for generating stimuli and an Acoustic Change Complex (ACC) measurement unit for measuring ACCs depending on the stimuli.

### TECHNICAL FIELD

The present invention relates to an apparatus for objectively detecting a cochlea dead region based on electrophysiology and to an information provision method for detecting the cochlea dead region using the apparatus.

### BACKGROUND ART

Recently, a plurality of problems related to congenital or acquired hearing loss has been raised. The problem of hardness of hearing (hearing impairment) attributable to rapid aging and industrialization has been identified as an issue to be solved in medical and welfare fields.

People of all age groups including the young generation as well as the old generation are frequently exposed to loud music or excessive levels of noise due to the development of portable Information Technology (IT) audio play devices, such as MP3 players or Portable Multimedia Players (PMPs), and the influence of a noisy environment. As a result, the rate of noise-induced sensorineural hearing loss has increased and become a social problem.

Cochlear hearing loss which is a representative phenomenon of hearing impairment is related to damage to the hair cells of the cochlea, and this damage causes hearing loss in the following two forms:

A first form is damage to outer hair cells, which is the cause of most sensorineural hearing loss. Due thereto, the active mechanism of the cochlea is damaged, so that the motion of a basilar membrane decreases compared to that of a normal state, with the result that frequency selectivity decreases. Speech recognition ability is relatively well maintained.

A second form is damage to inner hair cells. This may result in a decrease in the efficiency of signals transferred to a primary auditory cortex. In particular, speech recognition ability greatly decreases, and the ability to discriminate signals from noise is further deteriorated in the presence of noise. A typical hearing aid algorithm cannot improve the ability to discriminate signals from noise.

A region in which the inner hair cells are completely lost and do not perform their own functions is called a cochlea dead region (DR). The drawing and picture of the cochlea dead region are shown in FIGS. 1a and 1b (Brian C. J. Moore. (2009). Hearing Journal (Volume 62, Issue 3) 10-14.)

The cochlea dead region exhibits characteristics where the inner hair cells and nerves of the inside thereof do not induce nervous activities in response to stimuli falling within the range of relevant characteristic frequencies (CFs), and then relevant acoustic stimulus information is not transferred to a primary auditory cortex.

Cochlea dead regions can be classified as follows.

First, there is a high-frequency dead region. This has been most frequently reported, and mainly occurs at the basal end of the base of the cochlea. It is reported that damage attributable to aging and intense noise (for example, gunshots or explosions) is the most common cause of a high-frequency DR.

Second, there is a low-frequency dead region. This occurs less commonly, and occurs at the apical end of the base of the cochlea. It is reported that such a low-frequency DR occurs due to the advanced stages of Meniere's disease.

Last, there is a patchy dead region. This exhibits the characteristic of appearing in a specific part of the base of the cochlea. It is reported that the patchy dead region occurs due to various factors (for example, genetic factors, infection, autoimmune disease, exposure to toxic agents—anti cancer drugs, etc.).

The detection of dead regions in outer/inner hair cells of the base of the cochlea using conventional pure tone audiometry has experimental limitations. Therefore, as a new test method for detecting a cochlea dead region, a masking test method using Threshold-Equalizing Noise (TEN) (hereinafter referred to as a “TEN test”) is used. FIGS. 2a and 2b illustrate the spectra of the conventional TEN, and FIG. 3 illustrates a conventional TEN test apparatus.

The term “TEN” means a spectrum obtained based on the threshold of a pure tone to masking noise ranging from 250 Hz to 10000 Hz with respect to adults with normal hearing, and is produced based on Equivalent Rectangular Bandwidth Noise (ERB_{N}) of the auditory filters of normal-hearing persons.

A conventional TEN test apparatus and method will be described below with reference to FIG. 3. A TEN test is performed in a sequence similar to that of a conventional pure tone test normally performed in the ear, nose and throat department.

A Compact Disc (CD) sound source 11 in which TEN is recorded is prepared. The TEN recorded in the CD sound source 11 is replayed to an examinee 1 via a CD player 10 and an audiometer 12. The examinee 1 suitably responds to the TEN after hearing the TEN.

The level of the TEN is set to be at least 10 dB above a pure tone hearing threshold, and masked thresholds are obtained by adjusting the level of a pure tone in steps of 2 dBHL. Masked thresholds for respective frequencies (for example, 0.5, 1, 2, 4, and 8 kHz) are measured, are recorded on a masked audiogram, and are then compared. When the masked thresholds are at least 10 dBHL above absolute thresholds, a relevant region is determined to be a cochlea dead region.

Such a conventional TEN test apparatus and method performs a diagnosis relatively easily and simply, but this is subjective audiometry that depends on the behavioral response of the examinee to stimuli, and thus there is a considerable problem in the testing of the elderly or infants or children who cannot sufficiently understand the test procedure.

### DISCLOSURE OF INVENTION

### Technical Problem

Accordingly, the present invention has been made keeping in mind the above problems occurring in the prior art, and an object of the present invention is to provide a new apparatus and method, which can overcome the limitations of a conventional TEN test on the elderly or infants or children who cannot sufficiently understand the test procedure.

### Solution to Problem

In order to accomplish the above object, the present invention provides a Cochlea Dead Region (DR) detection apparatus, including a control unit which includes a stimulus generation unit for generating stimuli, and an Acoustic Change Complex (ACC) measurement unit for measuring ACCs depending on the stimuli.

Preferably, the each of the stimuli may be Threshold-Equalizing Noise (TEN). More preferably, each of the stimuli may include TEN noise and a pure tone. In particular, the pure tone may be contained in part of the TEN noise.

Preferably, the DR detection apparatus may further include an audiometer, a first end of which is connected to the stimulus generation unit and a second end of which is connected to a headphone; an amplifier, a first end of which is connected to the ACC measurement unit; and an output unit and an input unit for operating in conjunction with the control unit, wherein the output unit is capable of outputting the stimuli and the ACCs, measured depending on the stimuli, for respective frequencies, and the input unit is capable of selecting the stimuli for respective frequencies.

Further, in order to accomplish the above object, the present invention provides an information provision method for detecting a Cochlea Dead Region (DR), including (a) selecting stimuli for respective frequencies; (b) measuring Acoustic Change Complexes (ACCs) depending on the stimuli for respective frequencies; and (c) determining a cochlea dead region based on the ACCs measured for respective frequencies.

Preferably, (c) may include (c1) calculating absolute thresholds of the stimuli; (c2) calculating equalizing-noise masked thresholds of the ACCs measured for respective frequencies; and (c3) determining a cochlea dead region by comparing the absolute thresholds with the equalizing-noise masked thresholds for respective frequencies.

Preferably, (c3) may be configured such that a frequency region corresponding to a portion of the equalizing-noise masked thresholds which are at least 10 dBHL above the absolute thresholds is determined to be the cochlea dead region.

### Advantageous Effects of Invention

According to the present invention, the electrophysiology-based cochlea dead region detection apparatus and method is not yet present in domestic and foreign areas, and thus the present invention may be the only objective apparatus and method capable of detecting a cochlea dead region.

Further, since the present invention can present objective clinical data about both future clinical counseling and the application of cochlear implant surgery, which are related to compensation for hearing impairment of infants or children and the elderly who have difficulty in obtaining behavioral responses, it can be regarded as an apparatus and method capable of overcoming the limitations of a conventional test method based on behavioral responses.

Furthermore, the present invention can present an optimal hearing aid fitting method which utilizes a cochlea active region around a cochlea dead region in relation to the prescription of hearing aids.

Furthermore, when the apparatus and method of the present invention is applied, cochlea dead regions can be detected from a patient group having restrictions in a behavioral test, and clinical treatment and cure strategy based on this detection can be considered in various manners. In addition, the present invention can be clinically utilized for the counseling and hearing impairment compensation strategy for elderly hearing-impaired patients, and, in particular, the detection of cochlea dead regions of infants or children can provide an important clinical strategy access method for decision regarding the prescription of cochlear implant and hearing aids.

### BRIEF DESCRIPTION OF DRAWINGS

FIGS. 1a and 1b are diagrams showing a cochlea dead region;

FIGS. 2a and 2b are diagrams showing the spectra of TEN;

FIG. 3 is a diagram showing a conventional TEN test apparatus;

FIG. 4 is a diagram showing an ACC;

FIG. 5 is a diagram showing a TEN test apparatus according to the present invention;

FIG. 6 is a diagram showing the spectra of stimuli; and

FIGS. 7a to 7d are diagrams showing the results of the TEN test.

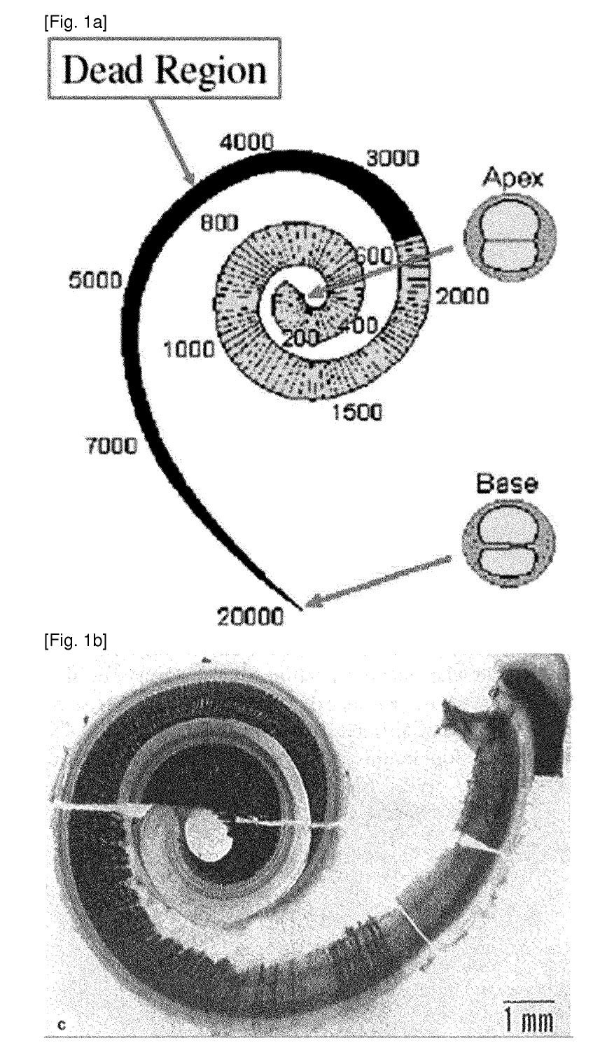

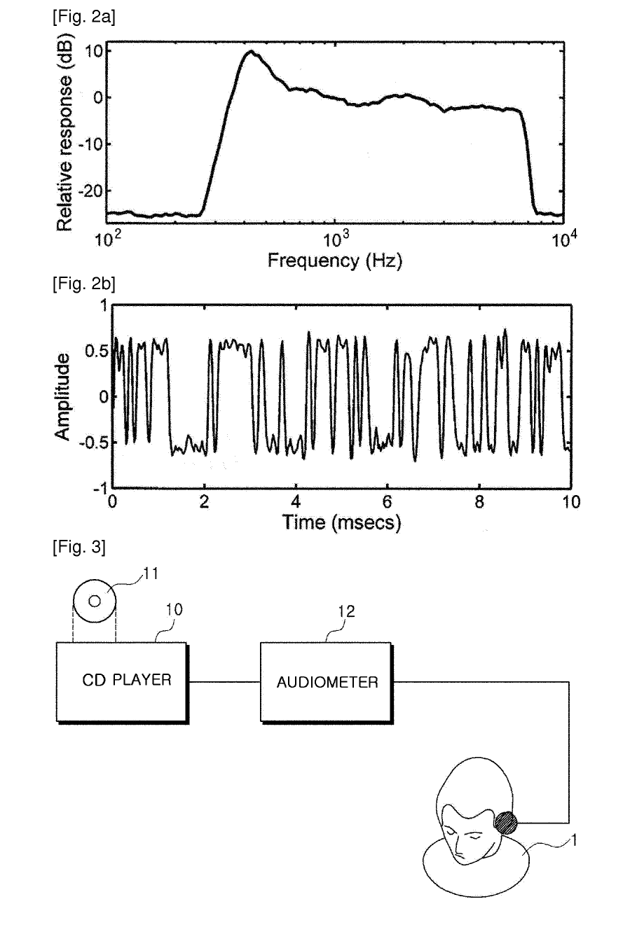

### MODE FOR THE INVENTION

Hereinafter, the present invention will be described in detail with reference to the attached drawings.

The present invention is intended to utilize an Acoustic Change Complex (ACC) to detect a cochlea dead region.

A P1-N1-P2 complex which is a portion of cortical auditory evoked potential is well known as a test method capable of determining the degree of hearing and neurogenic communication disorders using evoked neural responses to acoustic stimuli of normal-hearing persons and hearing-impaired persons.

An Acoustic Change Complex (ACC) has characteristics similar to those of a P1-N1-P2 complex, but is different in that a cortical response appears due to the change in the acoustic features of stimuli.

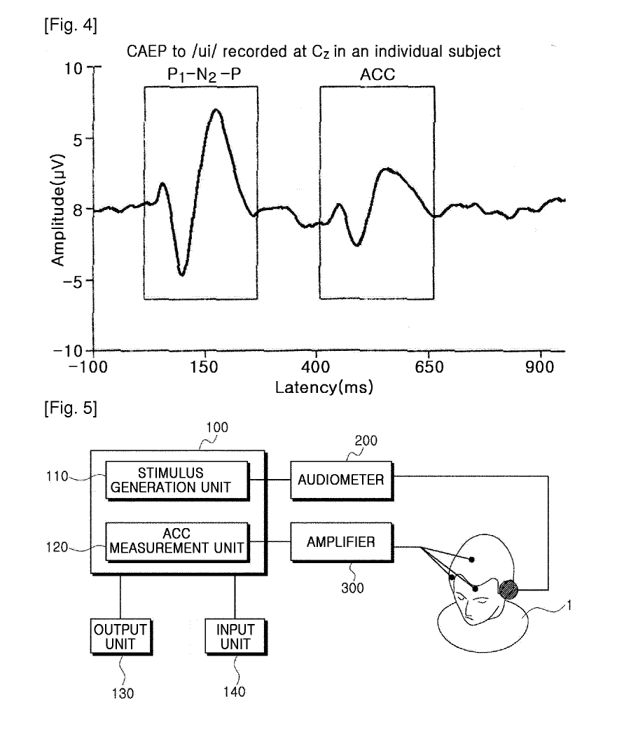

FIG. 4 is a graph showing a response to the sound /ui/. A first waveform responding to ‘/u/’ is represented by a P1-N1-P2 complex and a second waveform appearing at the change location ‘/i/’ of the progressing sound is represented by an ACC. In this way, the ACC responds very sensitively to the change in an acoustic parameter, such as the format of a spoken language, and the change in frequency and intensity, and is then closely related to the perceptivity of an examinee (Martin, B. A. & Boothroyd, A. (1999). Cortical, auditory, event-related potentials in response to periodic and aperiodic stimuli with the same spectral envelope. Ear and Hearing, 20(1), 33-44.).

Accordingly, the present invention is intended to propose a TEN test apparatus using an ACC.

Referring to FIG. 5, a TEN test apparatus according to the present invention will be described in detail.

A control unit 100 includes a stimulus generation unit 110 and an ACC measurement unit 120. The control unit 100 may be any medium capable of processing and storing data. It should be noted that the stimulus generation unit 110 and the ACC measurement unit 120 are merely classified according to the functions required for description and that there is no need to necessarily separate the functions thereof or information processing devices. Further, the “generation” of a stimulus (or stimuli) should be understood to be a concept including the “importing” of a stimulus through an external storage medium, as well as the generation of a stimulus by the control unit 100.

TEN, that is, a stimulus, is generated by the stimulus generation unit 110. The stimulus generated by the stimulus generation unit 110 can be replayed to an examinee 1 through an audiometer 200, a headphone, etc.

A TEN stimulus modified to measure electrophysiology is used as the stimulus. The stimulus is composed of TEN noise and a pure tone. For example, TEN noise required for the detection of a cochlea dead region based on electrophysiological test method has a duration of a total of two seconds, wherein a specific pure tone may not be contained in the TEN noise during the first second, and it may be contained in the TEN noise during the remaining one second. The response of the examinee can be measured while the level of a TEN noise-to-pure tone is changing in a range from 2 to 5 dBHL.

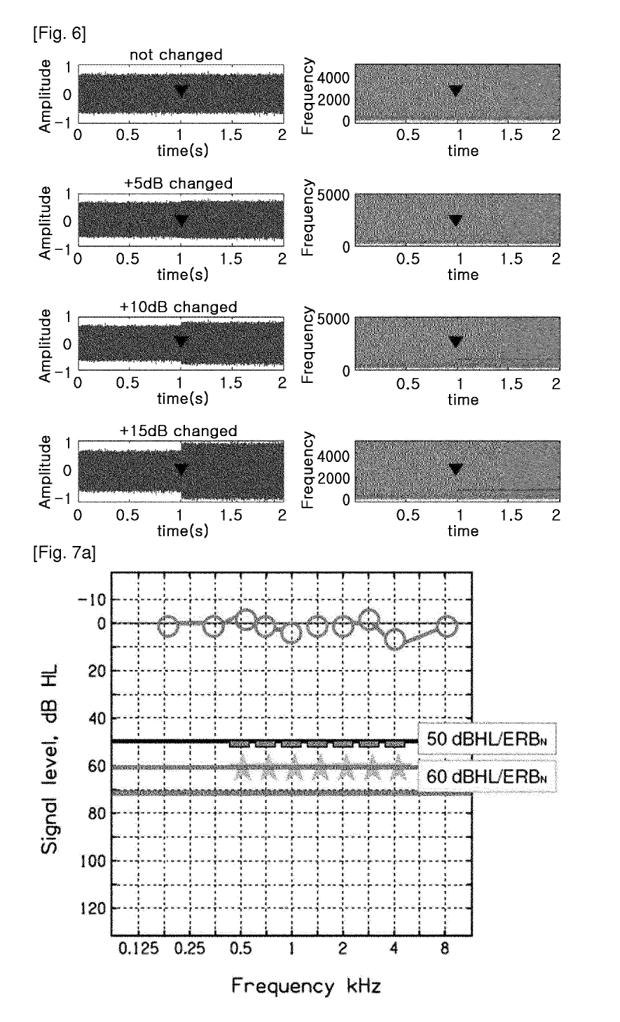

Examples of a stimulus will be described with reference to FIG. 6. In individual graphs, a black triangle denotes a pure tone, left graphs denote time versus amplitude groups, and right graphs denote time versus frequency graphs. A pure tone is contained in each stimulus after one second. Respective 1 kHz pure tones having levels of 0, +4, +8, and +12 dB in a direction from the uppermost to lowermost graphs are contained in TEN noise. It can be seen that as the pure tone level of the TEN noise increases, a 1 kHz frequency component is clearly indicated in a spectral domain after one second.

The ACC of the examinee 1 is measured by the ACC measurement unit 120 of the control unit 100 via an amplifier 300. Therefore, unlike the conventional TEN test, even if the examinee 1 does not overtly respond to the stimulus, an objective response can be measured.

The ACC measurement unit 120 can measure an ACC amplified by the amplifier 300 and can analyze the results of the measurement.

For example, a group of normal persons capable of behaviorally responding to a stimulus is tested while the conventional TEN test is performed, and then test data is obtained. Thereafter, the test data is analyzed based on an average comparison test (a paired t-test or a Wilcoxon's signed rank test) and a distribution comparison test, which are performed depending on the degree of variations in the results of behavioral and electrophysiological tests of a group of hearing-impaired examinees, thus enabling the test results of the examinees to be objectively obtained without requiring separate behavioral responses.

The control unit 100 operates in conjunction with an output unit 130 and an input unit 140. The output unit 130 can display stimuli used for tests or display the measured ACCs in the form of graphs. Via the input unit 140, stimuli can be selected or replayed to the examinee 1, or, alternatively, commands required to analyze test results can be selected. Further, the results of conventional pure tone audiometry can be input to the input unit 140 and can be output and compared together with the results obtained by the apparatus of the present invention.

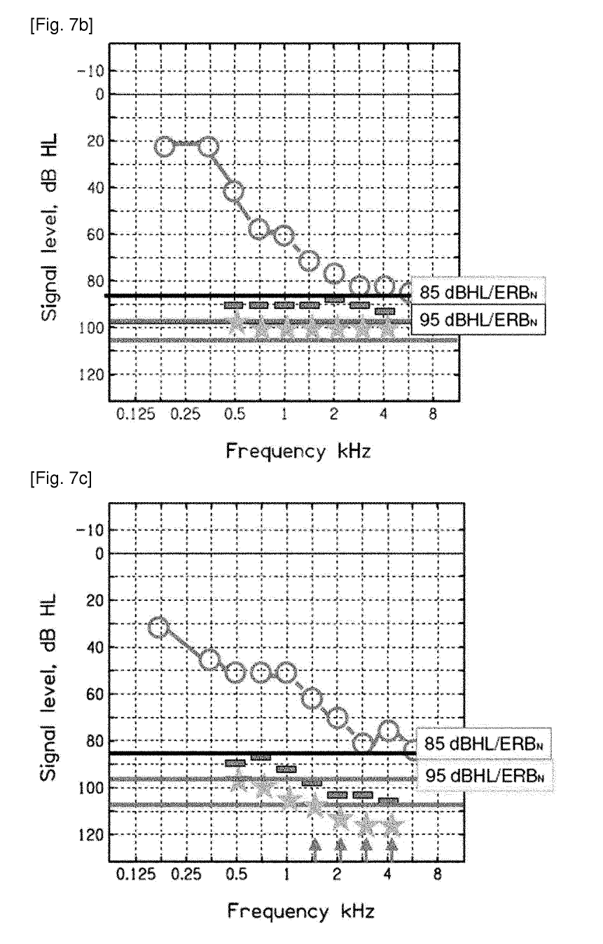

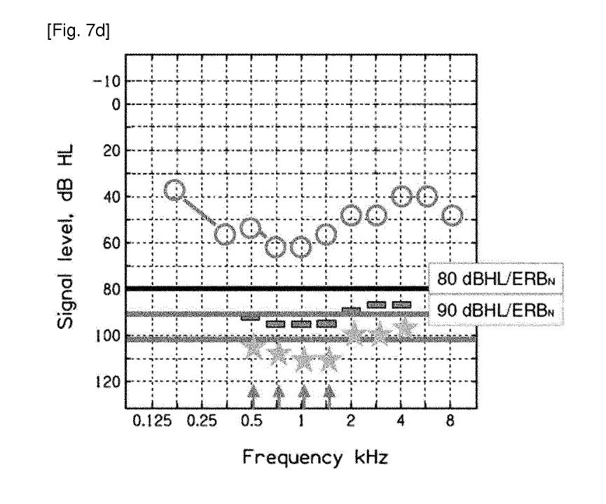

FIGS. 7a to 7d illustrate masking audiograms showing the results of a conventional behavioral TEN test, wherein those audiograms can be displayed through the output unit 130 using the apparatus according to the present invention.

In order to measure a cochlea dead region using the apparatus of the present invention, stimuli for respective frequencies are selected and prepared via the input unit 140. In this step, absolute thresholds of the stimuli for respective frequencies can be calculated.

Next, when the stimuli for respective frequencies are replayed to the examinee 1 by manipulating the control unit 100, the ACCs of the examinee 1 depending on the stimuli are measured by the ACC measurement unit 120. Equalizing-noise masked thresholds for respective frequencies can be calculated from the ACCs measured for respective frequencies.

Next, the absolute thresholds for respective frequencies are compared to the equalizing-noise masked thresholds for respective frequencies, and thus a cochlea dead region is determined. In more detail, a frequency region, corresponding to a portion of the equalizing-noise masked thresholds which are at least 10 dBHL above the absolute thresholds, is determined to be a cochlea dead region.

For example, if at high frequencies, the equalizing- noise masked thresholds are at least 10 dBHL above the absolute thresholds, and at low frequencies, they are not, it can be determined that a cochlea dead region is present in a high-frequency region and is not present in a low-frequency region.

Examples will be described with reference to FIGS. 7a to 7d. The test results of the present invention (green color in the lower portion of each graph) and the test results of the conventional pure tone audiometry (red circles in the upper portion of each graph) are shown together. The test results of the present invention include absolute thresholds calculated using stimuli and equalizing-noise masked thresholds analyzed using the measurement results of ACCs.

According to the conventional pure tone audiometry, it could be determined that the examinee of FIG. 7a was determined to be a normal-hearing person, and the examinees of FIGS. 7b to 7d were hearing-impaired persons.

Depending on the results of FIG. 7a, since there was no portion of equalizing-noise masked thresholds which are at least 10 dBHL above an absolute threshold (50 dBHL) (that is, equal to or greater than 60 dBHL), it was determined that, as a normal-hearing person, the examinee did not have a cochlea dead region.

Depending on the results of FIG. 7b, since there was no portion of equalizing-noise masked thresholds which are at least 10 dBHL above an absolute threshold (85 dBHL) (that is, equal to or greater than 95 dBHL), it was determined that, as a hearing-impaired person, the examinee did not have a cochlea dead region.

Depending on the results of FIG. 7c, since a portion of equalizing-noise masked thresholds which are at least 10 dBHL above an absolute threshold (85 dBHL) (that is, equal to or greater than 95 dBHL) could be detected at high frequencies (1.5 kHz or more), it was determined that the high-frequency region was a cochlea dead region (high-frequency cochlea dead region).

Depending on the results of FIG. 7d, since a portion of equalizing-noise masked thresholds which are at least 10 dBHL above an absolute threshold (80 dBHL) (that is, equal to or greater than 90 dBHL) could be detected at low frequencies (less than 1.5 kHz), it was determined that a low-frequency region was a cochlea dead region (low-frequency cochlea dead region).

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

### INDUSTRIAL APPLICABILITY

As described above, the electrophysiology-based cochlea dead region detection apparatus and method is not yet present in domestic and foreign areas, and thus the present invention may be the only objective apparatus and method capable of detecting a cochlea dead region.

1. A Cochlea Dead Region (DR) detection apparatus, comprising:
a control unit comprising,
a stimulus generation unit for generating stimuli; and
an Acoustic Change Complex (ACC) measurement unit for measuring ACCs depending on the stimuli.

2. The DR detection apparatus according to claim 1, wherein each of the stimuli is Threshold-Equalizing Noise (TEN).

3. The DR detection apparatus according to claim 2, wherein each of the stimuli comprises TEN noise and a pure tone.

4. The DR detection apparatus according to claim 3, wherein the pure tone is contained in part of the TEN noise.

5. The DR detection apparatus according to claim 1, further comprising:
an audiometer, a first end of which is connected to the stimulus generation unit and a second end of which is connected to a headphone;
an amplifier, a first end of which is connected to the ACC measurement unit; and
an output unit and an input unit for operating in conjunction with the control unit,
wherein the output unit is capable of outputting the stimuli and the ACCs, measured depending on the stimuli, for respective frequencies, and the input unit is capable of selecting the stimuli for respective frequencies.

6. An information provision method for detecting a Cochlea Dead Region (DR) using the DR detection apparatus according to claim 1, comprising:
(a) selecting stimuli for respective frequencies;
(b) measuring Acoustic Change Complexes (ACCs) depending on the stimuli for respective frequencies; and
(c) determining a cochlea dead region based on the ACCs measured for respective frequencies.

7. The information provision method according to claim 6, wherein (c) comprises:
(c1) calculating absolute thresholds of the stimuli;
(c2) calculating equalizing-noise masked thresholds of the ACCs measured for respective frequencies; and
(c3) determining a cochlea dead region by comparing the absolute thresholds with the equalizing-noise masked thresholds for respective frequencies.

8. The information provision method according to claim 7, wherein (c3) is configured such that a frequency region corresponding to a portion of the equalizing-noise masked thresholds which are at least 10 dBHL above the absolute thresholds is determined to be the cochlea dead region.
